(19) United States
(12) Patent Application Publication
(10) Pub. No.: US 2025/0221785 A1
(43) Pub. Date: Jul. 10, 2025

(54) THREE-DIMENSIONAL MAGNETIC MATTRESS FOR USE WITH RETAINED SURGICAL ITEM DETECTION SYSTEM

(71) Applicant: Covidien LP, Mansfield, MA (US)

(72) Inventor: Frank T. Smith, Clark, CO (US)

(21) Appl. No.: 18/853,460

(22) PCT Filed: Apr. 11, 2023
(86) PCT No.: PCT/IB2023/053684

§ 371 Date: Oct. 2, 2024

Related U.S. Application Data
(60) Provisional application No. 63/330,388, filed on Apr. 13, 2022.

(51) Int. Cl. A61B 90/00; A61B 90/98; A61G 13/10

(52) U.S. Cl. CPC A61B 90/08; A61B 90/98; A61G 13/10; A61B 2090/0804

(57) ABSTRACT

An interrogation and detection system for detection of surgical implements within a patient's body includes a signal generator configured to generate an energizing signal for an RF tag, and a surgical table. The surgical table includes a mattress disposed on the table, a first antenna, and a second antenna. The mattress defines a longitudinal axis. The first antenna and second antenna are operably coupled to the signal generator. The first antenna defines a first plane oriented in a first orientation relative to the longitudinal axis of the mattress. The second antenna defines a second plane that is oriented in a second orientation different than the orientation of the first plane of the first antenna.

### FIELD

This disclosure relates generally to interrogation and detection systems for radio-frequency (RF) tags, and more particularly, interrogation, detection and inventory systems for radio-frequency (RF) tags for use within surgical sites.

### BACKGROUND

It is often useful to determine whether objects associated with a surgery are present in a patient's body before completion of the surgery. Such objects may take a variety of forms. For example, the objects may take the form of instruments, for instance, scalpels, scissors, forceps, hemostats, and/or clamps. Also, for example, the objects may take the form of related accessories and/or disposable objects, for instance, surgical sponges, gauzes, and/or pads. Failure to locate an object before closing the patient may require additional surgery, and in some instances, may have unintended medical consequences.

Accordingly, there is a need for a technology that is capable of providing both presence detection and tagged surgical item/implement identification functionality in the medical setting, as well as inventory controls of the tagged items/implements. Specifically, detecting the presence of, identifying, and maintaining inventory of tagged surgical items and materials that are used during the execution of a medical procedure. Technologies exist that enable these functions both individually as well as in conjunction with each other, but the methods and packaging of the discrete solutions used are not ideal for the application. More specifically, the components attached or affixed to the items being tracked may either be too large physically and present nuisances or obstacles in the execution of the procedure, or the detection and identification performance of the solution may degrade rapidly in the presence of variable and uncontrolled dielectric or conductive materials.

Accordingly, there are needs for improvements in presence detection, tagged item identification, and inventory functionality in the medical setting.

### SUMMARY

This disclosure relates to systems for detection of surgical objects and devices used in body cavities during surgery, specifically a mattress for a surgical table with embedded antennae.

In accordance with aspects of the disclosure, an interrogation and detection system for detection of surgical implements within a patient's body is presented. The system includes a signal generator configured to generate an energizing signal for an RF tag, and a surgical table. The surgical table includes a mattress defining a longitudinal axis. A first antenna and a second antenna are operably coupled to the signal generator. The first antenna defines a first plane that is oriented in a first orientation relative to the longitudinal axis of the mattress. The second antenna defines a second plane that is oriented in a second orientation relative to the longitudinal axis of the mattress. The second orientation is different than the orientation of the first plane of the first antenna.

In an aspect of the present disclosure, the system may further include an RF tag configured to transmit a return signal when energized, the RF tag being affixed to a surgical implement. The first antenna may be configured to receive a return signal transmitted by the RF tag. The second antenna may be configured to receive the return signal transmitted by the RF tag.

In another aspect of the present disclosure, the first plane of the first antenna and the second plane of the second antenna may be oriented in a “V” like shape.

In yet another aspect of the present disclosure, the first antenna and the second antenna each may be disposed within the mattress.

In a further aspect of the present disclosure, the mattress may further include an array of antennae, each defining a plane and each plane having a unique orientation relative to the longitudinal axis of the mattress.

In yet a further aspect of the present disclosure, the first antenna and the second antenna may each include a coil antenna.

In an aspect of the present disclosure, the first antenna may generate a first magnetic field and the second antenna may generate a second magnetic field.

In another aspect of the present disclosure, the first antenna and the second antenna are each portion of a single antenna.

In yet another aspect of the present disclosure, the system may further include a processor and a memory. The memory may include instructions stored thereon, which, when executed by the processor, cause the system to determine whether a return signal was received from an RF tag that marks a surgical implement used in a procedure via at least one of the first antenna or second antenna.

In a further aspect of the present disclosure, the instructions, when executed by the processor, may further cause the system to transmit information to a display to display information related to the RF tag.

In accordance with aspects of the disclosure, a surgical mattress configured for detection of surgical implements within a body of a patient is presented. The surgical mattress includes a first antenna operably coupled to a signal generator and a second antenna operably coupled to the signal generator. The first antenna defines a first plane. The first plane of the first antenna is oriented in a first orientation relative to a longitudinal axis of the mattress. The second antenna defines a second plane. The second plane of the second antenna is oriented in a second orientation relative to the longitudinal axis of the mattress, the second orientation being different than the orientation of the first plane of the first antenna. In aspects, there may be multiple antennas in a plane. Each antenna can be individually operable in order to try to detect an RF tag

In yet a further aspect of the present disclosure, the surgical mattress may further include an RF tag configured to transmit a return signal when energized, the RF tag being affixed to a surgical implement. The first antenna may be configured to receive a return signal transmitted by the RF tag. The second antenna may be configured to receive the return signal transmitted by the RF tag.

In an aspect of the present disclosure, the first antenna and the second antenna may each include a coil antenna.

In another aspect of the present disclosure, the first antenna and the second antenna each may be disposed within the mattress.

In yet another aspect of the present disclosure, the first plane of the first antenna and the second plane of the second antenna may be oriented in a “V” like shape.

In yet another aspect of the present disclosure, the first antenna may generate a first magnetic field and the second antenna may generate a second magnetic field.

In a further aspect of the present disclosure, the first antenna and the second antenna are each portion of a single antenna.

In an aspect of the present disclosure, the surgical mattress may further include an array of antennae, each having a unique orientation.

In accordance with aspects of the disclosure, a computer-implemented method for detection of surgical implements within a body of a patient is presented. The method includes generating an energizing signal for an RF tag by a signal generator, the RF tag being affixed to a surgical implement, and receiving a return signal from the RF tag by at least one of a first antenna or a second antenna operably coupled to the signal generator. The first antenna defines a first plane oriented in a first orientation, and the second antenna defines a second plane oriented in a second orientation different than the orientation of the first plane of the first antenna.

In accordance with aspects of the disclosure, the computer-implemented method may further include determining whether a return signal was received from an RF tag that marks a surgical implement used in a procedure via at least one of the first antenna or second antenna. In yet a further aspect of the present disclosure, the computer-implemented method may further include transmitting information to a display to display information related to the RF tag.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged or shrunk and positioned to improve drawing legibility. Further, the particular shapes of the elements as drawn are not intended to convey any information regarding the actual shape of the particular elements and have been solely selected for ease of recognition in the drawings.

Various aspects of the presently disclosed antennae, RF tags, and articles containing them are described herein below with reference to the drawings.

FIG. 1 is a schematic diagram illustrating an interrogation and detection system configured for detecting an object within a patient that is tagged with an RF tag according to one illustrated aspect;

FIG. 2 is a functional block diagram of a controller configured for use with the interrogation and detection system of FIG. 1 in accordance with embodiments of the present disclosure;

FIGS. 3 and 4 are side cutaway views of surgical tables configured for use with the interrogation and detection system of FIG. 1 in accordance with embodiments of the present disclosure;

FIG. 5 is a side cutaway view of a magnetic mattress with embedded antennae for detection of surgical implements within a patient's body in active use within a surgical site in accordance with embodiments of the present disclosure; and

FIG. 6 is an illustration of a schematic illustration of an example implementation of an antenna for detection of surgical implements within a patient's body in active use within a surgical site.

### DETAILED DESCRIPTION

In the following description, certain specific details are set forth in order to provide a thorough understanding of disclosed aspects. However, one skilled in the relevant art will recognize that aspects may be practiced without one or more of these specific details or with other methods, components, materials, etc. In other instances, well-known structures associated with transmitters, receivers, or transceivers have not been shown or described in detail to avoid unnecessarily obscuring descriptions of the aspects.

Reference throughout this specification to “one aspect” or “an aspect” means that a particular feature, structure, or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects.

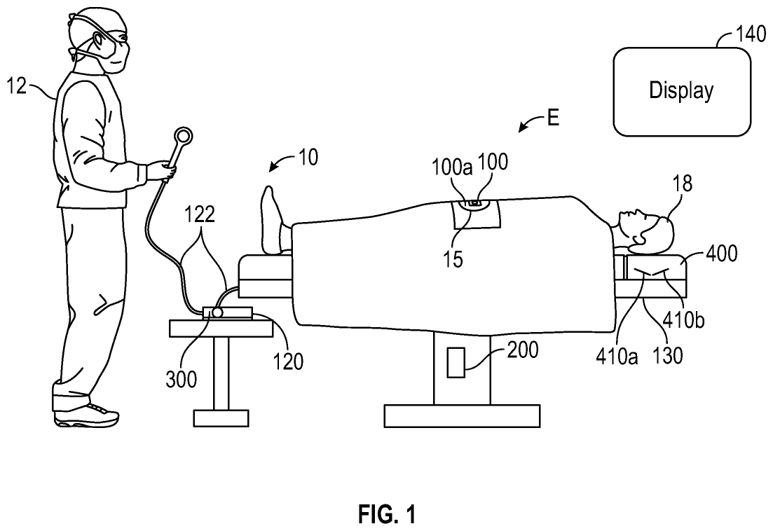

FIG. 1 depicts an interrogation and detection system 10 for detection of radio-frequency (RF) tags to ascertain the presence or absence of items, implements, or objects 100a in a patient 18. The interrogation and detection system 10 generally includes a signal generator 300 and antennae 410a, 410b coupled to the signal generator 300 by one or more communication paths, for example, coaxial cable 122. In one aspect of the interrogation and detection system 10, the antennae 410a, 410b may be embedded in a mattress 400. In aspects, the technology may utilize resonant tuned inductor and capacitor tags, and/or radio-frequency identification tags.

The object 100a may take a variety of forms, for example, instruments, accessories, and/or disposable objects useful in performing surgical procedures. For instance, the object 100a may take the form of scalpels, scissors, forceps, hemostats, and/or clamps. Also, for example, the objects 100a may take the form of surgical sponges, gauze, and/or padding. The object 100a is tagged, carrying, attached, or otherwise coupled to an RF tag 100. Aspects of the interrogation and detection system 10 disclosed herein are particularly suited to operate with one or more RF tags 100, which are not accurately tuned to a chosen or selected resonant frequency. Consequently, the RF tags 100 do not require high manufacturing tolerances or expensive materials and thus may be inexpensive to manufacture.

In use, the medical provider 12 may use the system 10 in order to detect the presence or absence of the one or more RF tags 100 and hence an object 100a in the patient 18. For a detailed description of an exemplary interrogation and detection system, reference may be made to commonly owned U.S. Patent Application Publication No. 2004/0250819 to Blair et al., titled “Apparatus and Method for Detecting Objects Using Tags And Wideband Detection Device,” filed Mar. 29, 2004, the entire contents of which is hereby incorporated by reference herein.

The term “read range,” as used in this disclosure, includes the distance from an antennae 410a, 410b (e.g., a reader coil) of an interrogation and detection system 10 and the geometric center of the RF tag 100 (FIG. 1). The term “orientation,” as used in this disclosure, includes the angle of incidence between the coil plane of the antenna 410a, 410b and the coil plane of the tag antenna or coil. Generally, for optimal orientation, the reader coil plane and the tag coil plane are parallel. Non-optimal orientation implies the primary reader coil plane, and the primary tag coil plane are not parallel, with the most challenging orientation being where the reader coil plane and the tag coil plane are orthogonal in space.

For typical RF applications, where communication between an interrogation and detection system 10 and an RF tag 100 (FIG. 1) is accomplished via coupled magnetic fields, the read range and sensitivity to coil orientation is a direct function of the coil areas and orientation. In other words, a larger coil will perform better in terms of the range where communication is possible, as well as the relative orientation between the antennae 410a, 410b and the RF tag coil. In the targeted application (detection and identification of tagged surgical items), the acceptable physical sizes of both the reader coil and the tag coil are smaller than those that would provide the optimal read range performance and reduced sensitivity to orientation. In the case of the RF tag 100 (FIG. 1), the acceptable physical size of the tracked tag component may be much smaller than ideal. In order to achieve an acceptable user experience in terms of both performance and physical form factors, novel methods of maximizing performance must be employed.

In aspects, the signal generator 300 may generate an energizing signal for an RF tag 100. The RF tag 100 may be affixed to a surgical implement. A first antenna 410a or a second antenna 410b, which are disposed in a mattress 400, located under a patient 18 may receive a return signal from the RF tag 100. The controller 200 may determine whether a return signal was received from an RF tag 100 that marks a surgical implement used in a procedure via at least one of the first antenna 410a or second antenna 410b. Then the controller may transmit information to a display 140 to display information related to the RF tag 100.

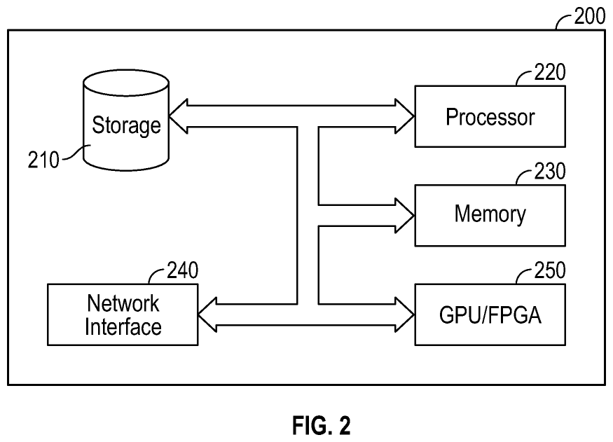

Now referring to FIG. 2, controller 200 includes a processor 220 connected to a computer-readable storage medium or a memory 230. The computer-readable storage medium or memory 230 may be a volatile type of memory, e.g., RAM, or a non-volatile type of memory, e.g., flash media, disk media, etc. In various aspects of the disclosure, the processor 220 may be another type of processor such as a digital signal processor, a microprocessor, an ASIC, a graphics processing unit (GPU), a field-programmable gate array (FPGA), or a central processing unit (CPU). In certain aspects of the disclosure, network inference may also be accomplished in systems that have weights implemented as memristors, chemically, or other inference calculations, as opposed to processors.

In aspects of the disclosure, the memory 230 can be random access memory, read-only memory, magnetic disk memory, solid-state memory, optical disc memory, and/or another type of memory. In some aspects of the disclosure, the memory 230 can be separate from the controller 200 and can communicate with the processor 220 through communication buses of a circuit board and/or through communication cables such as serial ATA cables or other types of cables. The memory 230 includes computer-readable instructions that are executable by the processor 220 to operate the controller 200. In other aspects of the disclosure, the controller 200 may include a network interface 240 to communicate with other computers or to a server. A storage device 210 may be used for storing data. The disclosed method may run on the controller 200 or on a user device, including, for example, on a mobile device, an IoT device, or a server system.

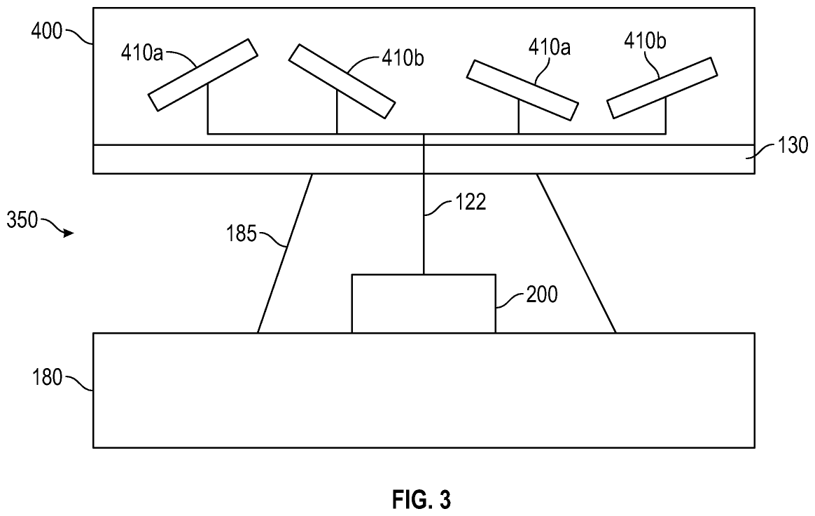

FIGS. 3 and 4 are side cutaway views of surgical tables configured for use with the interrogation and detection system of FIG. 1.

Referring to FIG. 3, the antennae 410a, 410b of the interrogation and detection system 10 are carried by the surgical table 350, for example, carried by inner layers of the mattress 130 or positioned in an interior of a shell or housing forming a patient support structure. The controller 200 of the interrogation and detection system 10 may be positioned in the pedestal or base 180, for example, in an interior of the pedestal or base 180. One or more wired or wireless communication paths may communicatively couple the controller 200 to the antennas 410a, 410b, and/or to an interrogation and detection system interface on display 140 (FIG. 1), for example, coaxial cable 122.

The interrogation and detection system 10 may receive power from a variety of sources, for example, from a wall outlet or receptacle via a conventional power cord and plug (not shown).

FIG. 4 shows an environment in which medical procedures are performed that include a bed (e.g., patient bed) 450 and an interrogation and detection system 1344, according to another illustrated embodiment. The aspects of FIG. 4 are similar in some respects to the previously described example, hence only significant differences in structure and operation will be discussed.

The environment may, for example, take the form of a hospital room, clinic room, or examination room of a medical practitioner's office.

The bed 450 may include a patient support structure 104 and a frame or base 458. The patient support structure 104 may support one or more mattresses, for example, a segmented mattress 400. The frame or base 458 may be made of plastic, metal, composite, reinforced composited, and/or roto-molded materials. Various commercially available designs of frames for patient beds are suitable. The frame or base 458 may include a set of wheels 112 (only one called out in FIG. 4), allowing the bed 450 to be easily moved. The frame or base 458 may include one or more rails 114, which may or may not be removable or which may or may not fold down.

The bed 450 may include one or more electric motors 108 and linkages 106, which are selectively actuated to move or articulate portions of the bed 450 or mattress 400. Other mechanisms may be used to move portions of the mattress 400. Commercially available patient or hospital beds 450 typically include one or more pieces of electrical or electronic equipment (e.g., electric motors 108) which are sources of radio noise that may interfere with the interrogation and detection system 10. Such equipment typically produces very consistent or periodic (i.e., non-random) noise. Some aspects of the interrogation and detection system 10 discussed herein employ various techniques to address such noise.

In aspects, antennae 410a, 410b of the interrogation and detection system 10 are carried by the patient support structure 104, mattress 400 or frame or base 458. For example, the antennae 410a, 410b may be carried by inner layers of the mattress 400 or positioned in an interior of a shell or housing forming the patient support structure 104. While illustrated as being in non-overlapping relationship, in some aspects the antennae 410a, 410b may overlap. A controller 200 of the interrogation and detection system 10 may be carried by the frame or base 458. One or more wired or wireless communication paths may communicatively couple the controller 200 to the antennae 410a, 410b and/or to an interrogation and detection system interface, for example, coaxial cable 122

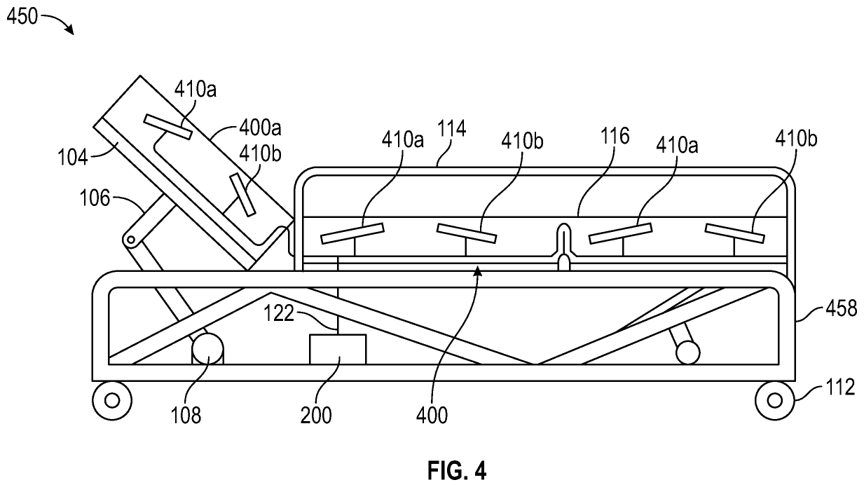

FIG. 5 is a side cutaway view of a mattress 400 with embedded antennae 410a, 410b for detection of surgical implements within a patient's body in active use within a surgical site. Mattresses 400 may typically be about 2 inches thick. However, any suitable thickness mattress 400 is contemplated.

The mattress 400 generally includes a first antenna 410a and a second antenna 410b, both disposed in the mattress 400. Antennae 410a, 410b are spaced along a longitudinal axis “X” of the mattress 400. The first antenna 410a and the second antenna 410b are both configured to be operably coupled to a signal generator 300 (FIG. 1). The first antenna 410aand the second antenna 410b are both further configured to receive a return signal transmitted by the RF tag 100 (FIG. 1). In aspects, the antennae 410a, 410b may include more than one pair of antennae.

The first antenna 410a and the second antenna 410b may include a coil antenna, a loop antenna, an antenna array, and/or any other suitable antenna arrangement. The first antenna 410a defines a first plane. The second antenna 410b defines a second plane.

In aspects, a magnetic field generated by the first antenna 410a may be positioned non-planar relative to a magnetic field generated by the second antenna 410b. For example, if the first antenna 410a is an array, the magnetic field may be non-orthogonal to the plane of the first antenna 410a. For example, the magnetic field of each antenna may be any suitable shape and/or direction.

The quality factor “Q” of an antenna directly influences the bandwidth of the antenna as well as the gain of the antenna. The interrogation and detection system 10 provides the benefit of using a higher “Q” antenna but being able to “capture” a wider area (than a similar system with planar-oriented antennae) because of the orientation of the antennae relative to each other.

The first plane defined by the first antenna 410a and the second plane defined by the second antenna 410b may be oriented in a “V” like shape. For example, the first antenna 410a may be oriented at an angle (angle ϕ3) of about 45 degrees relative to the second antenna 410b in the “V” like shape.

In aspects, the first plane of the first antenna 410a is oriented in a first orientation (e.g., at a first angle ϕ1 such as about 15 degrees) and the second plane of the second antenna 410b is oriented in a second orientation (e.g., at a second angle ϕ2 such as about 24 degrees) different than the orientation of the first antenna. The angle (ϕ1) of the first plane first antenna 410a and the angle (ϕ2) of the second plane of the second antenna 410b may each have a different angle relative to the longitudinal axis “X” of the mattress 400. Generally, when an RF tag is orthogonally oriented to an antenna's magnetic field, the antenna will not receive a signal. The interrogation and detection system 10 solves the problem of not receiving a response from the RF tag when the tag is orthogonally oriented to the antenna's magnetic field by orienting the antennae so that the antennae are not on the same plane. It is contemplated that any suitable non-planar orientation of antennae may be used. In aspects, the first antennae 410a may be oriented at a different orientation than the second antenna 410b.

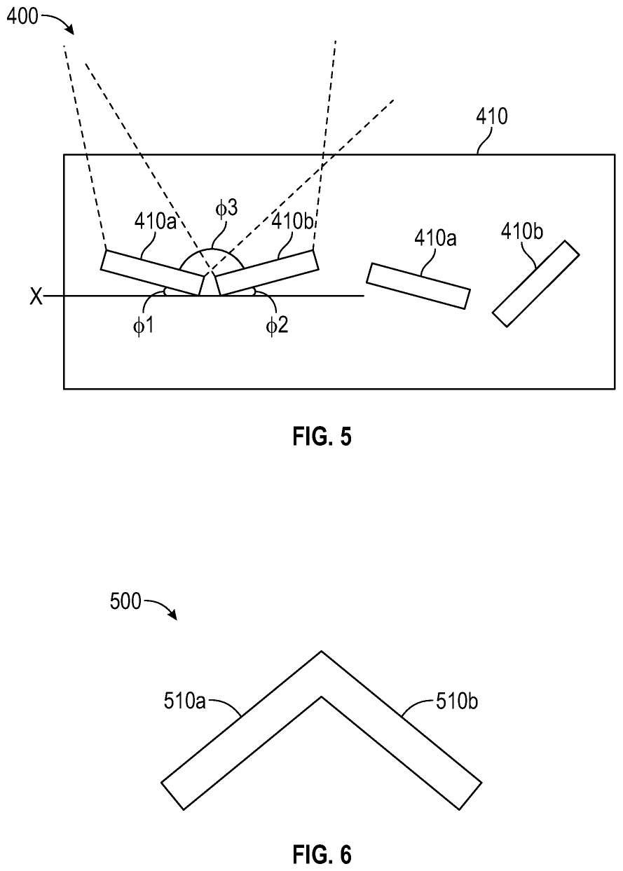

FIG. 6 is an illustration of a schematic illustration of an example implementation of an antenna for detection of surgical implements within a patient's body in active use within a surgical site. In aspects, a first antenna 510a and a second antenna 510b may be each portion of a single antenna 500. For example, the single antenna 500 may be a folded coil antenna.

While aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. An interrogation and detection system for detection of surgical implements within a body of a patient, the interrogation and detection system comprising:
a signal generator configured to generate an energizing signal for an RF tag; and
a surgical table including:
a mattress disposed on the surgical table, the mattress defining a longitudinal axis;
a first antenna operably coupled to the signal generator, the first antenna defining a first plane, wherein the first plane of the first antenna is oriented in a first orientation relative to the longitudinal axis of the mattress; and
a second antenna operably coupled to the signal generator, the second antenna defining a second plane, wherein the second plane of the second antenna is oriented in a second orientation different than the orientation of the first plane of the first antenna, the second orientation being relative to the longitudinal axis of the mattress.

2. The system of claim 1, further comprising an RF tag configured to transmit a return signal when energized, the RF tag affixed to a surgical implement,
wherein the first antenna is configured to receive a return signal transmitted by the RF tag, and
wherein the second antenna is configured to receive the return signal transmitted by the RF tag.

3. The system of claim 1, wherein the first plane of the first antenna and the second plane of the second antenna are oriented in a “V” like shape.

4. The system of claim 3, wherein the first antenna and the second antenna each are disposed within the mattress.

5. The system of claim 3, wherein the mattress further includes an array of antennae, each defining a plane and each plane having a unique orientation relative to the longitudinal axis of the mattress.

6. The system of claim 1, wherein the first antenna and the second antenna each include a planar coil antenna.

7. The system of claim 1, wherein the first antenna includes a first magnetic field and the second antenna includes a second magnetic field.

8. The system of claim 1, wherein the first antenna and the second antenna are each portion of a single antenna.

9. The system of claim 1, further comprising:
a processor; and
a memory, including instructions stored thereon, which, when executed by the processor, cause the system to:
determine whether a return signal was received from a RF tag that marks a surgical implement used in a procedure via at least one of the first antenna or second antenna.

10. The system of claim 9, wherein the instructions, when executed by the processor, further cause the system to:
transmit information to a display to display information related to the RF tag.

11. A surgical mattress configured for detection of surgical implements within a body of a patient, the surgical mattress comprising:
a first antenna operably coupled to a signal generator, the first antenna defining a first plane, wherein the first plane of the first antenna is oriented in a first orientation relative to a longitudinal axis of the mattress; and
a second antenna operably coupled to the signal generator, the second antenna defining a second plane, wherein the second plane of the second antenna is oriented in a second orientation different than the orientation of the first plane of the first antenna, the second orientation being relative to the longitudinal axis of the mattress.

12. The surgical mattress of claim 11, further comprising an RF tag configured to transmit a return signal when energized, the RF tag affixed to a surgical implement,
wherein the first antenna is configured to receive a return signal transmitted by the RF tag, and
wherein the second antenna is configured to receive the return signal transmitted by the RF tag.

13. The surgical mattress of claim 11, wherein the first antenna and the second antenna each include a coil antenna.

14. The surgical mattress of claim 11, wherein the first antenna and the second antenna each are disposed within the mattress.

15. The surgical mattress of claim 11, wherein the first plane of the first antenna and the second plane of the second antenna are oriented in a “V” like shape.

16. The surgical mattress of claim 11, wherein the first antenna includes a first magnetic field and the second antenna includes a second magnetic field.

17. The surgical mattress of claim 11, further comprising an array of antennae, each defining a plane and each plane having a unique orientation relative to the longitudinal axis of the mattress.

18. A computer-implemented method for detection of surgical implements within a body of a patient, the method comprising:
generating an energizing signal for an RF tag by a signal generator, the RF tag is affixed to a surgical implement; and
receiving a return signal from the RF tag by at least one of a first antenna or a second antenna operably coupled to the signal generator, the first antenna defining a first plane, wherein the first plane of the first antenna is oriented in a first orientation, the second antenna defining a second plane, wherein the second plane of the second antenna is oriented in a second orientation different than the orientation of the first plane of the first antenna.

19. The computer-implemented method of claim 18, further comprising:
determining whether a return signal was received from an RF tag that marks a surgical implement used in a procedure via at least one of the first antenna or second antenna.

20. The computer-implemented method of claim 18, further comprising:
transmitting information to a display to display information related to the RF tag.
